(19) United States
(12) Patent Application Publication
(10) Pub. No.: US 2021/0397649 A1
(43) Pub. Date: Dec. 23, 2021

(54) RECOGNITION APPARATUS, RECOGNITION METHOD, AND COMPUTER-READABLE RECORDING MEDIUM

(71) Applicant: NEC Corporation, Minato-ku, Tokyo (JP)

(72) Inventors: Shivangi MAHTO, Tokyo (JP); Takayuki ARAKAWA, Tokyo (JP)

(73) Assignee: NEC Corporation, Minato-ku, Tokyo (JP)

(21) Appl. No.: 17/289,536

(22) PCT Filed: Oct. 29, 2018
(86) PCT No.: PCT/JP2018/040183

§ 371 Date: Apr. 28, 2021

(51) Int. Cl. G06F 16/65; G06F 21/32; A61B 5/12; A61B 5/00; A61B 5/117

(52) U.S. Cl. CPC G06F 16/65; G06F 21/32; A61B 5/117; A61B 5/6817; A61B 5/126

(57) ABSTRACT

A recognition apparatus 100 for ear acoustic recognition include a feature normalizer 101 which reads input ear acoustic data and removes the earphone's resonance effect from the input ear acoustic data to produce a normalized data at the output, a feature extractor 102 which extracts acoustic features from the normalized data, a classifier 103 which reads the acoustic features as input and classifies them into their corresponding class.

### TECHNICAL FIELD

The present invention relates to a recognition apparatus, a recognition method for ear acoustic recognition, and also to a computer-readable recording medium having recorded thereon a pattern recognition program for realizing the apparatus or the method.

### BACKGROUND ART

Ear acoustic biometrics refers to the biometric authentication of a person by the means of ear canal acoustics. The acoustic properties of the pinna and ear canal have been proven to be unique for each person and hence, can be used as a characteristic to differentiate among individuals.

To capture the ear acoustics of an individual, a probe sound signal is transmitted from an earphone device to the ear canal of the individual and an echo signal is recorded through the microphone integrated into the earphone. Then, using the probe and echo signals, ear acoustics for the individual is extracted for the recognition purpose. The technology in ear acoustic biometrics uses a pattern recognition system to recognize a person using his captured ear acoustics.

Pattern recognition has been widely used in various spheres of life including day-to-day applications such as security, surveillance, and e-commerce as well as technological applications such as agriculture, engineering, science and high-profile issues like military and national security.

Processes of a pattern recognition system can be broadly categorized into two steps: The first is feature extraction to extract features of an input signal, and the second is classification to classify the extracted features into a class (classes) corresponding to the input signal. In the case of ear acoustic biometrics, the input signal is the captured ear acoustic and the predicted classes are labels corresponding to the recognized users.

The pattern recognition system learns features corresponding to the classes and trains its classifier using learnt features. For better pattern recognition, features should have class related properties and should not depend on other external features for e.g., type of channel used for recording the input signal and noise. Dependency on the type of channel and noise results in larger within-class variability for an individual.

In real world scenarios, the type of ear phone used for capturing the ear acoustics of an individual often affects the performance of feature extraction and classification processes. Due to resonance effect of the earphones, ear acoustics can get corrupted and the expected property of features of being independent of nature of earphones cannot be satisfied. This dependency on the nature of earphones also creates mismatch among features of an individual captured using different kind of earphones and hence results in poor recognition performance.

One approach to keep the above mentioned expected property of features in a pattern recognition apparatus is to apply a feature normalization block to handle generalized unwanted variability in features introduced by dependency on type of earphones. The block is desired to make within-class variance or covariance in multi-dimensional cases as small as possible relative to between-class covariance by transforming features into another feature space. It is expected to remove the resonance effect of the earphones from the captured ear acoustic of individuals so as to minimize within-class variability.

To handle the problem of increased within-class variance and/or decreased between-class variance in the feature space due to distortion in input signal caused by earphone, a feature normalization has been applied to the extracted features before classification. The normalization has the property to remove the resonance effect of the earphone from the captured acoustics of individual.

A prior art for this method is disclosed in PTL1 as shown in FIG. 8. FIG. 8 is a block diagram of prior art.

As shown in FIG. 8, a feature extractor reads captured ear acoustics data as input (x) and extract acoustic features such as Mel-frequency Cepstral Coefficients (MFCCs) from the data as (z). Classifier such as LDA/PLDA reads the extracted features as input (z) and estimates their class labels (l).

Objective function calculator reads original labels of the input feature (o) and estimated class labels by classifier (l). It calculates cost of the classification as classification error between original labels (l) and estimated class labels (o). Parameter updater updates parameters of the classifier according to the minimization of cost function. This process keeps going on till convergence. After convergence, parameter updater stores parameters of the classifier in storage.

In test phase, feature extractor reads input test ear acoustic data, assuming that the same earphone is used to capture the acoustic data as for the training data and produces its acoustic features. Then classifier reads structure and parameters from the storage. Then it reads the acoustic features as input and predict their corresponding their classes.

The PTL1 shows limitation on handling the ear acoustic data of individuals captured by the means of more than one kind of earphones. It constraints that the training and test data must belong to same kind of earphone. Also, it does not handle the effect of earphone resonance on the captured ear acoustics.

Above described method does not handle the within-class variability introduced in the captured ear acoustics of an individual due to different nature of earphones used for capturing. The domain mismatch between training and test data due to different earphones results in poor recognition performance and restricts the users to use same earphone every time.

### CITATION LIST

### Patent Literature

[PTL 1]
WO2017069118 A1

### SUMMARY OF INVENTION

### Technical Problem

A summary of the technical challenges and the solution provided by the inventive technique will be represented next.

To handle within-class variability and noise, a robust pattern recognition system is very important. Distortion in input ear acoustic signal due to earphone's resonance effect and other factors can cause large within-class covariance relative to between-class covariance in feature space which results in worse pattern recognition accuracy.

One of the important properties of features for good pattern recognition is to have small within-class covariance relative to between-class covariance. Features should not have any dependency on the nature of the earphone and its resonance effect.

To handle the resonance effect of earphone in the ear acoustic data, it is conceivable to remove the resonance effect from acoustic data with the help of the label of the earphone used for capturing the data and a dictionary of resonance of various kind of earphones.

However, the prior art that disclosed in PTL 1 does not handle the within-class variability introduced by the variety of earphones used for capturing the ear acoustics data. It constraints the user to use same earphone for test and train purpose.

One example of an object of the present invention is to resolve the above problems and provide a recognition apparatus, recognition method, and a computer-readable recording medium that can remove the resonance effect of earphone from acoustic data.

In addition to the entities mentioned above, other apparent and obvious drawbacks that this invention can overcome will be revealed from the detailed specification and drawings.

### Solution to Problem

In order to achieve the foregoing object, a recognition apparatus according to one aspect of the present invention includes:

a feature normalizer that reads input ear acoustic data and removes the earphone's resonance effect from the input ear acoustic data to produce a normalized data at the output;

a feature extractor that extracts acoustic features from the normalized data;

a classifier that reads the acoustic features as input and classifies them into their corresponding class.

In order to achieve the foregoing object, a recognition method according to another aspect of the present invention includes:

(a) a step of reading input ear acoustic data and removing the earphone's resonance effect from the input ear acoustic data to produce a normalized data at the output;

(b) a step of extracting acoustic features from the normalized data;

(c) a step of reading the acoustic features as input and classifies them into their corresponding class.

In order to achieve the foregoing object, a computer-readable recording medium according to still another aspect of the present invention has recorded therein a program for ear acoustic recognition by a computer, and the program includes an instruction to cause the computer to execute:

(a) a step of reading input ear acoustic data and removing the earphone's resonance effect from the input ear acoustic data to produce a normalized data at the output;

(b) a step of extracting acoustic features from the normalized data;

(c) a step of reading the acoustic features as input and classifies them into their corresponding class.

### Advantageous Effects of Invention

An advantage of the invention is that we get a trained feature normalization block with the desired properties of features as follows:

It collects the acoustic resonance of various kind earphones by utilizing the nature of acoustic resonance of a hollow tube.

It removes the acoustic resonances of the earphones from the captured ear acoustics of individuals. Hence, helps to decrease with-in class variability and get better representation of the ear acoustic features.

The added block helps in achieving better classification accuracy.

The invention accordingly comprises of several steps and the relation of one or more of these steps with respect to each of the others, and the apparatus embodying the features of construction, combinations of elements and arrangement of parts that are adapted to affect such steps, all will be exemplified in the following detailed disclosure, i.e. description of drawings and detailed description. The scope of the invention will be indicated in the claims.

### BRIEF DESCRIPTION OF DRAWINGS

The drawings together with the detailed description, serve to explain the principles for the inventive method. The drawings are for illustration and do not limit the application of the technique.

FIG. 1 is a block diagram illustrating the schematic configuration of the recognition apparatus according to an example of primary embodiment of the present invention.

FIG. 2 is a block diagram illustrating the specific configuration of the recognition apparatus according to the embodiment of the present invention divide into a training stage and trial stage: the training of classifier in the ear recognition system using normalized ear acoustic data.

FIG. 3 is a block diagram illustrating 2-step processing of the feature normalizer shown in FIG. 2. First step is preparation of earphone's resonance directory for further usage in the ear recognition system and second step is how the block performs while using the recognition.

FIG. 4 is a flowchart illustrating operations of training stage performed by the recognition apparatus according to the embodiment of the present invention: training of a classifier with the help of normalized ear acoustics data

FIG. 5 is a flowchart illustrating operations of classification in trail stage performed by the recognition apparatus according to the embodiment of the present invention. FIG. 5 shows performing classification using the trained classifier.

FIG. 6 is a flowchart illustrating operations of transformation in trail stage performed by the recognition apparatus according to the embodiment of the present invention. FIG. 6 shows the use of trained matric of the classifier for features transformation to obtain discriminative features.

FIG. 7 is a block diagram showing an example of a computer that realizes the recognition apparatus according to the embodiment of the present invention.

FIG. 8 is a block diagram of prior art—the current state-of-the-art ear acoustic recognition system which constraints to use same kind of earphone to be used during training and test stage.

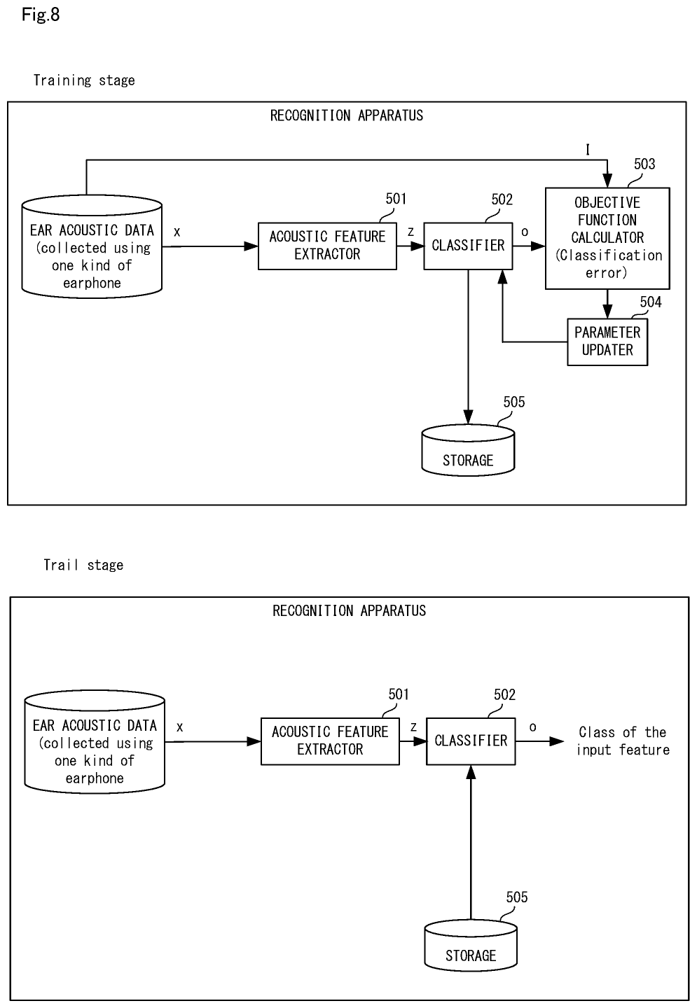

### DETAILED DESCRIPTION

Principle of the Invention

A summary of the solution to all these problems is provided next. To solve the technical problems discussed above an overall approach is summarized here. There are two stages in the approach as a training stage and a test stage.

In training stage, a feature normalization block reads the training ear acoustic data and produces normalized data as output by removing earphone's resonance effect. Acoustic feature extractor reads the normalized data as input and extracts corresponding acoustic features.

Classifier reads the extracted features as input and estimates their class labels. Objective function calculator reads original labels of the input feature and estimated class labels by classifier. It calculates cost of the classification as classification error between original labels and estimated class labels.

Parameter updater updates parameters of the classifier according to the minimization of cost function. This process keeps going on till convergence. After convergence, parameter updater stores parameters of the classifier in storage.

In test stage, the feature normalization block reads the given test acoustic data and produces normalized data. Then, feature extractor reads the normalized data as input and extracts corresponding acoustic features. Following this, classifier reads the extracted acoustic features as input and predicts the corresponding class.

The feature normalization block consists of 2-step processing. First step is to prepare a dictionary of acoustic resonances of various kind of earphones. This step is done before using the block in the ear acoustic recognition system.

In this step, first a collector collects the acoustic responses of a hollow cylindrical tube with the help of a mic-integrated earphone by transmitting white noise. Secondly, a separator performs source separation on each of the recorded acoustic responses of hollow tube to separate the resonances of earphone from that of captured hollow tube using signal processing for e.g., Non-negative Matrix Factorization source separation. Thirdly, the storage stores the separated acoustic resonance of the earphone in the dictionary with the type of earphone as the label.

The second step of the block is performed in the system during both of the training and test stage for the normalization of the input ear acoustic features. In this step, a resonance remover reads the input ear acoustic data and the type of earphone used to capture it.

Then, it looks up for the acoustic resonance of the used earphone from the dictionary prepared in Step 1. After that, the remover removes the earphone's resonance from the input data and gives the normalized data as output. Direct subtraction or some source separation techniques can be used by the remover for removal purpose.

### Embodiment

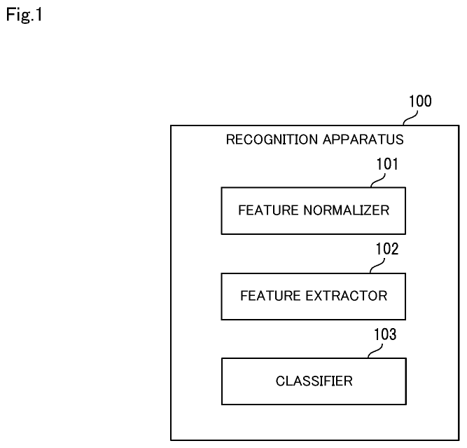

Hereinafter, a recognition apparatus, a recognition method, and a program of exemplary embodiments of the present invention will be described in detail with reference to FIGS. 1 to 6. The implementations are described in complete detail. Along with the illustrative drawings, the explanation provided here is so as to provide a solid guide to a person skilled in the art to practice this invention.

### Device Configuration

First, the schematic configuration of the recognition apparatus of the embodiment will be described. FIG. 1 is a block diagram illustrating the schematic configuration of the recognition apparatus according to the embodiment of the present invention.

A recognition apparatus 100 of the embodiment shown in FIG. 1 is an apparatus for ear acoustic recognition. As shown in FIG. 1, the recognition apparatus 100 includes a feature normalizer 101, a feature extractor 102, and a classifier 103.

The feature normalizer 101 reads input ear acoustic data and removes the earphone's resonance effect from the input ear acoustic data to produce a normalized data at the output. The feature extractor 102 extracts acoustic features from the normalized data. The classifier reads the acoustic features as input and classifies them into their corresponding class.

In this way, with the recognition apparatus 100, the resonance effect of earphone is removed from acoustic data. For this reason, it is possible to improved pattern recognition accuracy

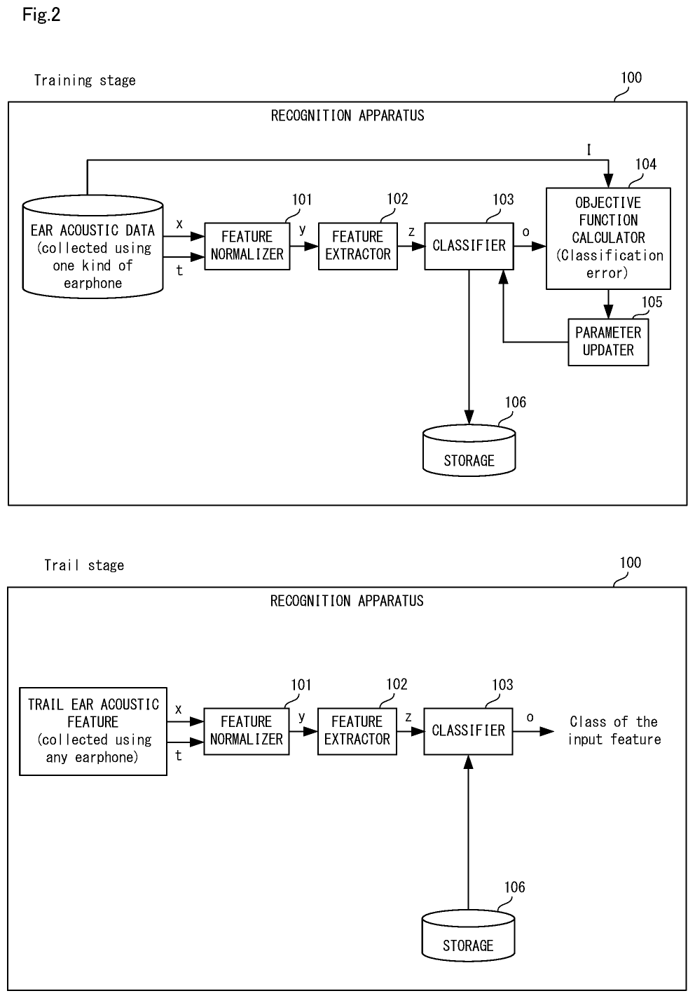

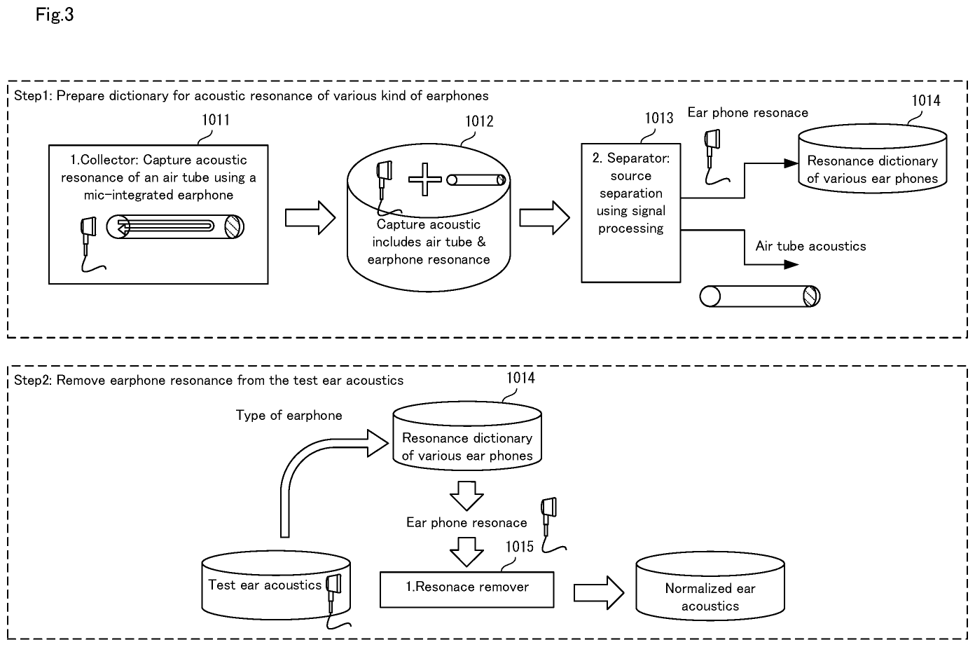

Next, the configuration of the recognition apparatus 100 of the embodiment will be described in detail with reference to FIGS. 2 and 3 as well.

FIG. 2 is a block diagram illustrating the specific configuration of the recognition apparatus according to the embodiment of the present invention divided into a training stage and a trial stage.

As shown in FIG. 2, the recognition apparatus further includes an objective function calculator 104 which calculates classification error as the cost function, a parameter updater 105, and a storage 106 which stores structure and parameters of classifier 103, in addition to the feature normalizer 101, the feature extractor 102, and the classifier 103.

In the training stage, the feature normalizer 101 reads captured ear acoustic data x and the type of earphone used for capturing the data t. Then, the feature normalizer 101 looks up for the resonance of earphone t and removes it from the input ear acoustic features and produces the normalized ear acoustic data y at the output.

The feature extractor 102 reads normalized acoustic data y as input and extracts acoustic features z at output. The classifier 103 receives the extracted acoustic features z as input and classifies them into their corresponding classes o. The classifier can be any classifier such as support vector machines or neural networks.

The objective function calculator 104 calculates cost 1041 as classification error 1042 between estimated classes of input features o and original labels of classes 1. The parameter updater 105 updates parameters of classifier according to cost minimization. This process keeps going till convergence when no more cost function can be reduced. After convergence, the parameter updater 105 stores parameters of the trained classifier in the storage 106.

In the trial stage, the feature normalizer 101 reads the input test data x′ and produces normalized data as output y′. The feature extractor 102 reads normalized data as input and extracts corresponding feature at output z′. The classifier 103 reads its stored structure and parameters from the storage 106. The classifier 103 reads test acoustic features as input and predicts its class at the output o′.

FIG. 3 is a block diagram illustrating 2-step processing of the feature normalizer 101 shown in FIG. 2. As shown in FIG. 2, the feature normalizer 101 includes a collector 1011, a storage 1012, a separator 1013, a storage 1014, and a resonance remover 1015. The feature normalizer 101 executes a two-step process.

First step is preparation process of resonance directory using the collector 1011 which collects acoustic resonance of a hollow tube in storage 1012, the separator 1013, and the Storage 1014. Second step process is resonance removal using the resonance remover 1015.

In first step, the collector 1011 collects the acoustic responses of a hollow cylindrical tube with the help of a mic-integrated earphone by transmitting white noise and stores it in Storage 1012.

Then, the separator 1013 performs source separation on each of the recorded acoustic responses of hollow tube to separate the resonances of earphone from that of captured hollow tube using signal processing for e.g., Non-negative Matrix Factorization source separation (NMF).

NMF reads spectrogram of input captured acoustic data and perform source separation on to produce 2 spectrograms at output corresponding to 2 sources. One source is common among all the input that is the hollow tube air resonance and another source is the earphone's acoustic resonance. This separated acoustic resonance of the earphone is stored in the dictionary with the type of earphone as the label in the storage 1014.

In second step, the resonance remover 1015 reads the input ear acoustic data and the type of earphone used to capture it. Then, the resonance remover 1015 looks up for the acoustic resonance of the used earphone in the Storage 1014 consisting of the resonance dictionary.

After that, the resonance remover 1015 removes the obtained earphone's resonance from the input data and gives the normalized data as output. Direct subtraction or some source separation techniques can be used by the remover for removal purpose. Spectrograms of ear acoustics are taken as input.

### Device Operation

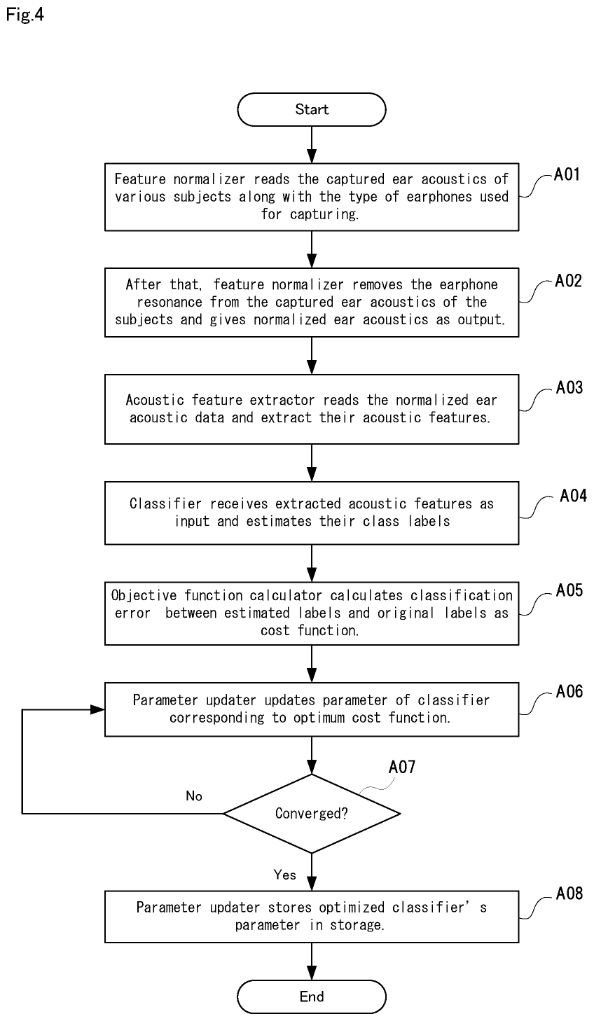

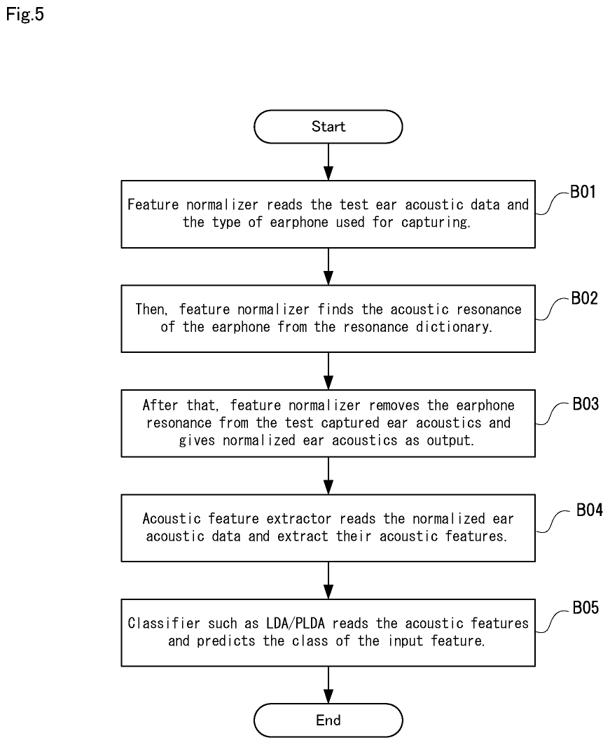

Next, operations performed by the recognition apparatus 100 of the embodiment will be described with reference to FIGS. 4, 5 (a) and 5 (b). Also, in the embodiment, a recognition method is implemented by causing the recognition apparatus to operate. Accordingly, the following description of operations performed by the recognition apparatus 100 will substitute for a description of the recognition method of the present embodiment.

First, with reference to FIG. 4, the training stage will be described FIG. 4 is a flowchart illustrating operations of training stage performed by the recognition apparatus according to the embodiment of the present invention.

In training stage, the feature normalizer 101 reads the training ear acoustic data and the type of earphone used to capture the data (step A01). Next, the feature normalizer 101 produces normalized data as output by removing earphone's resonance effect (step A02). Next, the acoustic feature extractor 102 reads the normalized data as input and extracts corresponding acoustic features (step A03).

Then, the classifier 103 reads the extracted features as input and estimates their class labels (step A04). Next, the objective function calculator 104 reads original labels of the input feature and estimated class labels by classifier. The objective function calculator 104 calculates cost of the classification as classification error between original labels and estimated class labels (step A05).

Then, the parameter updater 105 updates the parameters of the classifier 103 according to the minimization of cost function (step A06). The parameter updater 105 keeps going to execute step A06 until the parameters of the classifier 103 convergence (step A07). After convergence, the parameter updater 105 stores the parameters of the classifier 103 in storage 106 (step A08).

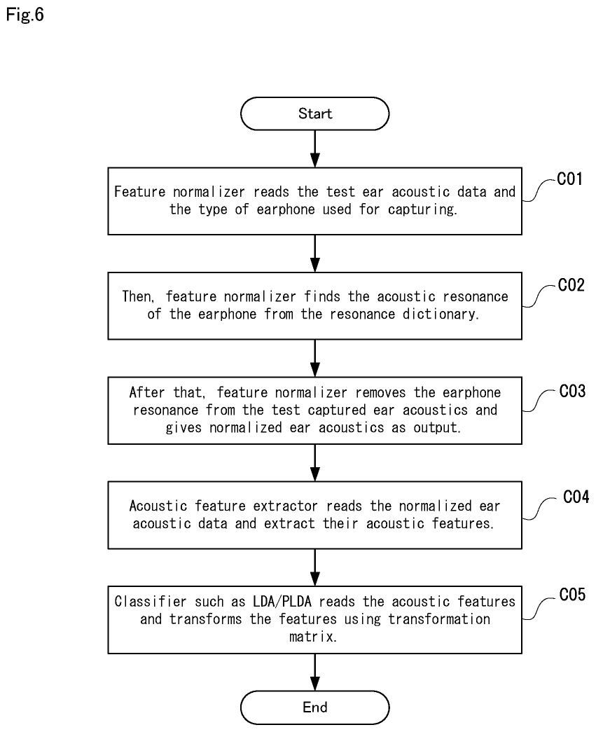

Next, with reference to FIGS. 5 and 6, the trail stage will be described These figures show the 2 kinds of possible trail stage of the embodiment. First flow chart FIG. 5 demonstrates classification of ear acoustic data using trained classifier. FIG. 5 is a flowchart illustrating operations of classification in trail stage performed by the recognition apparatus according to the embodiment of the present invention.

As shown in FIG. 5, first, the feature normalizer 101 reads the input test data and the type of earphone (step B01). Then, the feature normalizer 101 finds the acoustic resonance of the earphone from the resonance dictionary (step B02). Next, the feature normalizer 101 removes the earphone resonance from the input acoustic data and produces normalized data as output (step B03).

Next, the acoustic feature extractor 102 reads normalized data as input and extracts corresponding feature at output (step B04). After that, the classifier 103 reads its stored structure and parameters from the storage 106. The classifier 103 reads test acoustic features as input and predicts its class at the output (step B05).

Second flow chart FIG. 6 demonstrates discriminative feature extraction from ear acoustic data using trained classifier. FIG. 6 is a flowchart illustrating operations of transformation in trail stage performed by the recognition apparatus according to the embodiment of the present invention.

As shown in FIG. 6, first, the feature normalizer 101 reads the input test data and the type of earphone (step C01). Then, the feature normalizer 101 finds the acoustic resonance of the earphone from the resonance dictionary (step C02). Next, the feature normalizer 101 removes the earphone resonance from the input acoustic data and produces normalized data as output (step C03).

Next, the acoustic feature extractor 102 reads normalized data as input and extracts corresponding feature at output (step C04). Then, the classifier 103 reads its stored structure and parameters from the storage. Next, the classifier 103 reads test acoustic features as input and transforms them using its trained matrix to discriminative features. (step C05).

### Program

It is sufficient that the program of the embodiment is a program for causing a computer to execute steps A01 to A08 shown in FIG. 4, steps B01 to B05 shown in FIG. 5 and steps C01 to C05 shown in FIG. 6. The recognition apparatus 100 and the recognition method of the embodiment can be realized by the program being installed in the computer and executed. In this case, a processor of the computer functions as and performs processing as the feature normalizer 101, the feature extractor 102, the classifier 103, the objective function calculator 104, and the parameter updater 105.

Note that the program of the embodiment may be executed by a computer system that is constituted by multiple computers. In this case, the computers may respectively function as the feature normalizer 101, the feature extractor 102, the classifier 103, the objective function calculator 104, and the parameter updater 105, for example.

### Physical Configuration

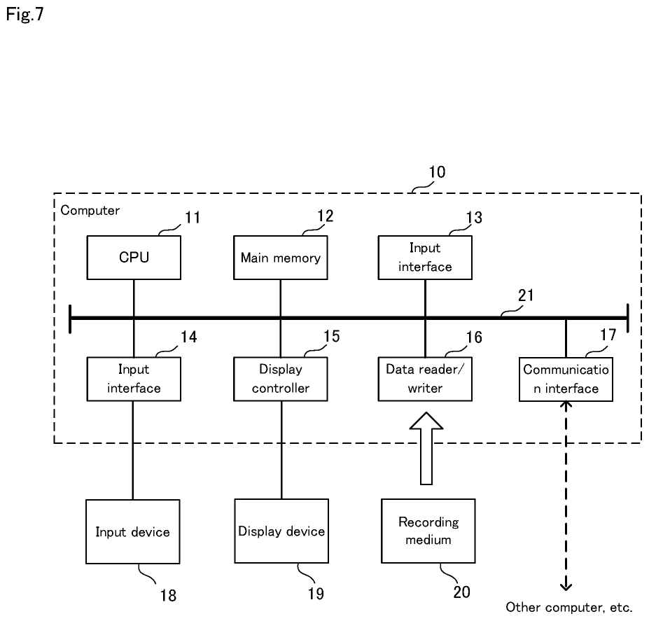

The following describes a computer that realizes the recognition apparatus by executing the program of the embodiment, with reference to FIG. 7. FIG. 7 is a block diagram showing an example of a computer that realizes the recognition apparatus according to the embodiment of the present invention.

As shown in FIG. 7, a computer 10 includes a CPU (Central Processing Unit) 11, a main memory 12, a storage device 13, an input interface 14, a display controller 15, a data reader/writer 16, and a communication interface 17. These units are connected by a bus 21 so as to be able to communicate with each other.

The CPU 11 deploys programs (code) of this embodiment, which are stored in the storage device 13, to the main memory 12, and executes various types of calculation by executing the programs in a predetermined order. The main memory 12 is typically a volatile storage device such as a DRAM (Dynamic Random-Access Memory). The programs of this embodiment are provided in a state of being stored in a computer-readable recording medium 20. Note that the programs of this embodiment may be distributed over the Internet, which is accessed via the communication interface 17.

Other specific examples of the storage device 13 include a hard disk and a semiconductor storage device such as a flash memory. The input interface 14 mediates the transfer of data between the CPU 11 and an input device 18 such as a keyboard or a mouse. The display controller 15 is connected to a display device 19, and controls screens displayed by the display device 19.

The data reader/writer 116 mediates the transfer of data between the CPU 11 and the recording medium 20 and executes the readout of programs from the recording medium 20, and the writing of processing results obtained by the computer 10 to the recording medium 20. The communication interface 17 mediates the transfer of data between the CPU 11 and another computer.

Specific examples of the recording medium 20 include a general-purpose semiconductor storage device such as a CF (Compact Flash (registered trademark)) card or an SD (Secure Digital) card, a magnetic storage medium such as a Flexible Disk, and an optical storage medium such as a CD-ROM (Compact Disk Read Only Memory).

Note that the recognition apparatus of the above embodiments can also be realized by using hardware that corresponds to the various units, instead of a computer in which a program is installed. Furthermore, a configuration is possible in which a portion of the recognition apparatus is realized by a program, and the remaining portion is realized by hardware.

Part or all of the embodiments described above can be realized by Supplementary Notes 1 to 15 described below, but the present invention is not limited to the following descriptions.

### (Supplementary Note 1)

A recognition apparatus for ear acoustic recognition comprising:

a feature normalizer that reads input ear acoustic data and removes the earphone's resonance effect from the input ear acoustic data to produce a normalized data at the output;

a feature extractor that extracts acoustic features from the normalized data;

a classifier that reads the acoustic features as input and classifies them into their corresponding class.

### (Supplementary Note 2)

The recognition apparatus according to supplementary note 1,

wherein the feature normalizer reads the input ear acoustic data along with the type of earphone used for capturing the input ear acoustic data, searches the earphone's acoustic resonance in a dictionary of acoustic resonances of various earphone, removes the searched earphone's resonance from the input ear acoustic data, and produces the normalized ear acoustic data at the output.

### (Supplementary Note 3)

The recognition apparatus according to supplementary note 2,

wherein the acoustic resonances of earphones in the dictionary are made by capturing acoustic responses of a hollow tube with the earphones attached in it and separating the acoustic resonances of the earphones from the one of the hollow tube.

### (Supplementary Note 4)

The recognition apparatus according to supplementary note 3,

wherein the acoustic resonances of earphones are obtained by blind source separation that extracts signal components which are common over earphones and signal components which are unique to individual earphones from captured acoustic responses.

### (Supplementary Note 5)

The recognition apparatus according to supplementary note 4,

wherein the acoustic resonances of earphones are obtained by using non-negative matrix factorization as a blind source separation technique.

### (Supplementary Note 6)

A recognition method for ear acoustic recognition comprising:

(a) a step of reading input ear acoustic data and removing the earphone's resonance effect from the input ear acoustic data to produce a normalized data at the output;

(b) a step of extracting acoustic features from the normalized data;

(c) a step of reading the acoustic features as input and classifies them into their corresponding class.

### (Supplementary Note 7)

The recognition method according to supplementary note 6,

wherein in the step (a), reading the input ear acoustic data along with the type of earphone used for capturing the input ear acoustic data, searching the earphone's acoustic resonance in a dictionary of acoustic resonances of various earphone, removing the searched earphone's resonance from the input ear acoustic data, and producing the normalized ear acoustic data at the output.

### (Supplementary Note 8)

The recognition method according to supplementary note 7,

wherein in the step (a), the acoustic resonances of earphones in the dictionary are made by capturing acoustic responses of a hollow tube with the earphones attached in it and separating the acoustic resonances of the earphones from the one of the hollow tube.

### (Supplementary Note 9)

The recognition method according to supplementary note 8,

wherein in the step (a), the acoustic resonances of earphones are obtained by blind source separation that extracts signal components which are common over earphones and signal components which are unique to individual earphones from captured acoustic responses.

### (Supplementary Note 10)

The recognition method according to supplementary note 9,

wherein in the step (a), the acoustic resonances of earphones are obtained by using n-negative matrix factorization as a blind source separation technique.

### (Supplementary Note 11)

A computer-readable medium having recorded thereon a program for ear acoustic recognition by a computer, the program including instructions for causing the computer to execute:

(a) a step of reading input ear acoustic data and removing the earphone's resonance effect from the input ear acoustic data to produce a normalized data at the output;

(b) a step of extracting acoustic features from the normalized data;

(c) a step of reading the acoustic features as input and classifies them into their corresponding class.

### (Supplementary Note 12)

The computer-readable medium according to supplementary note 11,

wherein in the step (a), reading the input ear acoustic data along with the type of earphone used for capturing the input ear acoustic data, searching the earphone's acoustic resonance in a dictionary of acoustic resonances of various earphone, removing the searched earphone's resonance from the input ear acoustic data, and producing the normalized ear acoustic data at the output.

### (Supplementary Note 13)

The computer-readable medium according to supplementary note 12,

wherein in the step (a), the acoustic resonances of earphones in the dictionary are made by capturing acoustic responses of a hollow tube with the earphones attached in it and separating the acoustic resonances of the earphones from the one of the hollow tube.

### (Supplementary Note 14)

The computer-readable medium according to supplementary note 13,

wherein in the step (a), the acoustic resonances of earphones are obtained by blind source separation that extracts signal components which are common over earphones and signal components which are unique to individual earphones from captured acoustic responses.

### (Supplementary Note 15)

The computer-readable medium according to supplementary note 14,

wherein in the step (a), the acoustic resonances of earphones are obtained by using n-negative matrix factorization as a blind source separation technique.

As a final point, it should be clear that the process, techniques and methodology described and illustrated here are not limited or related to a particular apparatus. It can be implemented using a combination of components. Also, various types of general purpose devise may be used in accordance with the instructions herein. The present invention has also been described using a particular set of examples.

However, these are merely illustrative and not restrictive. For example, the described software may be implemented in a wide variety of languages such as C++, Java, Python and Perl etc. Moreover, other implementations of the inventive technology will be apparent to those skilled in the art.

### INDUSTRIAL APPLICABILITY

According to the present invention, it is possible to remove the resonance effect of earphone from acoustic data. The present invention is useful in ear acoustic recognition.

### REFERENCE SIGNS LIST

10 Computer
11 CPU
12 Main memory
13 Storage device
14 Input interface
15 Display controller
16 Data reader/writer
17 Communication interface
18 Input device
19 Display apparatus
20 Storage medium
21 Bus
100 Recognition apparatus
101 Feature normalizer
102 Feature extractor
103 Classifier
104 Objective function calculator
105 Parameter updater.
106 Storage
1011 Collector
1012 Storage
1013 separator
1014 storage
1015 resonance remover

What is claimed is:

1. A recognition apparatus for ear acoustic recognition comprising:
a feature normalizer that reads input ear acoustic data and removes the earphone's resonance effect from the input ear acoustic data to produce a normalized data at the output;
a feature extractor that extracts acoustic features from the normalized data;
a classifier that reads the acoustic features as input and classifies them into their corresponding class.

2. The recognition apparatus according to claim 1,
wherein the feature normalizer reads the input ear acoustic data along with the type of earphone used for capturing the input ear acoustic data, searches the earphone's acoustic resonance in a dictionary of acoustic resonances of various earphone, removes the searched earphone's resonance from the input ear acoustic data, and produces the normalized ear acoustic data at the output.

3. The recognition apparatus according to claim 2,
wherein the acoustic resonances of earphones in the dictionary are made by capturing acoustic responses of a hollow tube with the earphones attached in it and separating the acoustic resonances of the earphones from the one of the hollow tube.

4. The recognition apparatus according to claim 3,
wherein the acoustic resonances of earphones are obtained by blind source separation that extracts signal components which are common over earphones and signal components which are unique to individual earphones from captured acoustic responses.

5. The recognition apparatus according to claim 4,
wherein the acoustic resonances of earphones are obtained by using non-negative matrix factorization as a blind source separation technique.

6. A recognition method for ear acoustic recognition comprising:
reading input ear acoustic data and removing the earphone's resonance effect from the input ear acoustic data to produce a normalized data at the output;
extracting acoustic features from the normalized data;
reading the acoustic features as input and classifies them into their corresponding class.

7. The recognition method according to claim 6,
wherein in the reading, reading the input ear acoustic data along with the type of earphone used for capturing the input ear acoustic data, searching the earphone's acoustic resonance in a dictionary of acoustic resonances of various earphone, removing the searched earphone's resonance from the input ear acoustic data, and producing the normalized ear acoustic data at the output.

8. The recognition method according to claim 7,
wherein in the reading, the acoustic resonances of earphones in the dictionary are made by capturing acoustic responses of a hollow tube with the earphones attached in it and separating the acoustic resonances of the earphones from the one of the hollow tube.

9. The recognition method according to claim 8,
wherein in the reading, the acoustic resonances of earphones are obtained by blind source separation that extracts signal components which are common over earphones and signal components which are unique to individual earphones from captured acoustic responses.

10. The recognition method according to claim 9,
wherein in the reading, the acoustic resonances of earphones are obtained by using n-negative matrix factorization as a blind source separation technique.

11. A non-transitory computer-readable medium having recorded thereon a program for ear acoustic recognition by a computer, the program including instructions for causing the computer to execute:
reading input ear acoustic data and removing the earphone's resonance effect from the input ear acoustic data to produce a normalized data at the output;
extracting acoustic features from the normalized data;
reading the acoustic features as input and classifies them into their corresponding class.

12. The non-transitory computer-readable medium according to claim 11,
wherein in the reading the input ear acoustic data along with the type of earphone used for capturing the input ear acoustic data, searching the earphone's acoustic resonance in a dictionary of acoustic resonances of various earphone, removing the searched earphone's resonance from the input ear acoustic data, and producing the normalized ear acoustic data at the output.

13. The non-transitory computer-readable medium according to claim 12,
wherein in the reading, the acoustic resonances of earphones in the dictionary are made by capturing acoustic responses of a hollow tube with the earphones attached in it and separating the acoustic resonances of the earphones from the one of the hollow tube.

14. The non-transitory computer-readable medium according to claim 13,
wherein in the reading, the acoustic resonances of earphones are obtained by blind source separation that extracts signal components which are common over earphones and signal components which are unique to individual earphones from captured acoustic responses.

15. The non-transitory computer-readable medium according to claim 14,
wherein in the reading, the acoustic resonances of earphones are obtained by using n-negative matrix factorization as a blind source separation technique.
